(19) United States
(12) Patent Application Publication
(10) Pub. No.: US 2017/0303810 A1
(43) Pub. Date: Oct. 26, 2017

(54) Capacitive Electrocardiography (ECG) Physiological Monitoring Systems

(71) Applicant: Medical Design Solutions, Inc., Milpitas, CA (US)

(72) Inventor: Robert T. Stone, Sunnyvale, CA (US)

(21) Appl. No.: 15/491,775

(22) Filed: Apr. 19, 2017

Related U.S. Application Data
(60) Provisional application No. 62/325,290, filed on Apr. 20, 2016.

(51) Int. Cl. A61B 5/0408; A61B 5/0205; A61B 5/04; A61B 5/00; A61B 5/0404; A61B 5/044

(52) U.S. Cl. CPC A61B 5/04085; A61B 5/044; A61B 5/0205; A61B 5/6893; A61B 5/6804; A61B 5/0006; A61B 5/0404; A61B 5/04012; A61B 2562/0214; A61B 2562/182

(57) ABSTRACT

An electrocardiography (ECG) physiological monitoring system that includes an ECG sensor assembly having first and second capacitive electrodes that are configured to electrically couple to a subject's skin and detect ECG signals, a reference electrode that is configured to average the capacitance potential of the first and second electrodes, an electronics module that is in direct communication with the ECG sensor assembly and programmed to control the ECG sensor assembly, process ECG signals therefrom, and wirelessly transmit the processed ECG signals, and transmission conductors that are configured to provide a signal communication path between the electronics module and the ECG sensor assembly.

### FIELD OF THE INVENTION

The present invention relates to systems and methods for monitoring physiological characteristics of a subject. More particularly, the present invention relates to systems and methods for determining physiological characteristics as a function of electrocardiography (ECG) characteristics signals.

### BACKGROUND OF THE INVENTION

As is well known in the art, ECG signals provide an effective means for determining one or more physiological characteristics of a mammalian subject. By monitoring the ECG signals of a subject, any deviation in a seminal physiological characteristic, e.g. heart rate, can be detected and immediately transmitted to a physician for analysis and prompt preventative action(s). ECG is thus one of the most widely used means for monitoring the health status of a subject.

Conventional clinical ECG systems typically employ 12-15 silver-silver chloride (Ag—AgCl) electrodes, i.e. wet ECG electrodes, which are affixed to specific regions of the chest, arms, or hands and legs. Wet ECG electrodes provide a wet conducting medium for charge transfer between the electrodes and the body. Adhesive tape is also often applied to keep the wet ECG electrodes in place.

There are, however, several drawbacks and disadvantages associated with wet ECG electrodes and, hence, ECG systems employing same. A major drawback is that wet electrode ECG systems often require cleaning of the attachment site on the body and, in some instances, also shaving hair off the attachment site. Cleaning of the attachment site is inconvenient and can cause skin irritation, allergic reactions, and inflammation due to toxicological issues of the electrode gels in long-term treatments.

Additionally, the quality of the signal will be compromised as the electrode gel dehydrates during prolonged use.

Further, since it is difficult to keep the electrode gel and/or adhesives entirely separate from each other over the long term. Cross-coupling between neighboring electrode sites can thus occur through leakage current.

In an effort to overcome the drawbacks associated with wet electrode ECG systems, gel-less, i.e. dry, ECG electrode systems have been developed. Dry electrode ECG systems typically employ 4-12 capacitive electrodes and a reference electrode.

There are, however, similarly several drawbacks and disadvantages associated with dry ECG electrodes and, hence, ECG systems employing same.

A major drawback associated with dry ECG electrode systems is that the amplitude levels of ECG signals detected by a dry electrode are typically very low. Thus, detecting spontaneous changes in ECG signals is very difficult.

Further, the high impedance exhibited by dry ECG electrodes necessitates an on-site amplifier with ultra-high impedance. Since the level of electrical noise introduced by the on-site amplifier is proportional to the magnitude of the impedances, signal quality of the amplifier output is degraded.

Dry ECG electrode systems also often exhibit poor signal quality due to external signal interference; particularly, electromagnetic radiation.

Dry electrode systems are also subject to internal signal interference that is generated by changes in the displacement of the electrode-to-skin distance and friction between the electrodes and the medium that the electrodes are attached to, e.g. fabric of a garment.

A further drawback associated with dry ECG electrode systems is that the systems typically employ a remote reference electrode that is positioned in such a manner that the body of a subject is disposed between the dry ECG electrode(s) and reference electrode.

By way of example, one conventional ECG system, known as the Electric Potential Integrated Circuit (EPIC) sensor system, which is produced by Plessey Semiconductors Ltd. (Application Note 291566, Issue No. 1), is designed and configured to be employed in a seat. The Plessey EPIC sensor system employs a large reference electrode that is incorporated into the seat and two ECG sensors that are incorporated into the clothing of an individual. To receive ECG signals with the Plessey EPIC sensor system, a subject must be positioned in the signal path between the reference electrode and the two ECG sensors.

Although the Plessey EPIC sensor system reduces external signal interference, internal signal interference is amplified.

Another major drawback of the EPIC sensor system is that the system only utilizes the “best” or “cleanest” ECG signal that is detected by one of the ECG sensors, while discarding the ECG signal that is detected by the other ECG electrode. As a result, the EPIC sensor system provides an ECG signal having substantially reduced accuracy due to the inherent subjectivity of the signal processing method.

It would thus be desirable to provide an improved ECG physiological monitoring system and method that (i) accurately monitors ECG signals representing one or more physiological characteristics associated with a user or wearer, (ii) does not require the use of any conductive gels, (iii) is suitable for long term monitoring of physiological characteristics, (iv) substantially reduces or abates external electromagnetic interference and (v) substantially reduces or abates internal interference.

It is therefore an object of the present invention to provide an improved ECG physiological monitoring system and method that (i) accurately monitors ECG signals representing one or more physiological characteristics associated with a user or wearer, (ii) does not require the use of any conductive gels, (iii) is suitable for long term monitoring of physiological characteristics, (iv) substantially reduces or abates external electromagnetic interference and (v) substantially reduces or abates internal interference.

It is another object of the present invention to provide an ECG physiological monitoring system and method that is suitable for long term monitoring of physiological characteristics.

It is another object of the present invention to provide an ECG physiological monitoring system and method that includes reliable and effective means to connect external modules, e.g. processing units.

### SUMMARY OF THE INVENTION

The present invention is directed to an improved system and method for determining physiological characteristics as a function of electrocardiography (ECG) characteristics signals.

In one embodiment of the invention, the ECG physiological monitoring system includes an ECG sensor system, signal transmission conductors and an electronics module.

In some embodiments, the ECG sensor system further includes first and second ECG electrodes and a reference electrode.

In a preferred embodiment, the first and second ECG electrodes comprise capacitive electrodes that are designed and configured to detect ECG signals without direct physical communication with the skin of a subject.

According to the invention, the reference electrode provides a capacitive reference as a function of the capacitance potential of the first and second ECG electrodes. In a preferred embodiment, the capacitive reference comprises the average of the capacitance potential of the first and second ECG electrodes (C_{Avg}).

In some embodiments, the reference electrode is also configured to average the capacitance potential noise generated by the anatomy of a subject, e.g., skin, muscle, etc., and the capacitance potential of the first and second ECG electrodes to provide an average capacitance potential value (C′_{Avg}).

In a preferred embodiment, the average capacitance potential values C_{Avg}, C′_{Avg} represent the ECG signal associated with the subject.

In some embodiments, the reference electrode provides an electromagnetic shield for the first and second ECG electrodes.

In some embodiments, the physiological monitoring system further includes an electronics module that preferably includes a processing system, which is programmed and configured to control the ECG sensor system and the function thereof, and the transmission and receipt of signals therefrom.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of one embodiment of an ECG physiological monitoring system, in accordance with the invention;

FIG. 2 is a top plane view of one embodiment of an ECG sensor system, in accordance with the invention;

FIG. 3 is a perspective view of one embodiment of a wearable ECG physiological monitoring system employing the ECG sensor system shown in FIG. 2, in accordance with the invention;

FIG. 4 is a perspective view of one embodiment of an external ECG physiological monitoring system, in accordance with the invention;

FIG. 5A is a top plane view of another embodiment of an ECG sensor system shown in FIG. 4, in accordance with the invention;

FIG. 5B is a top plane view of another embodiment of an ECG sensor system shown in FIG. 4, in accordance with the invention; and

FIG. 6 is a perspective view of another embodiment of an external ECG physiological monitoring system, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a sensor signal” includes two or more such signals and the like.

### Definitions

The term “monitor”, as used herein, means and includes, without limitation, one-time and continuous detection of ECG signal(s) and, hence, physiological characteristics and parameters of a subject associated therewith over a defined period of time.

The terms “physiological parameter” and “physiological characteristic”, as used herein, mean and include, without limitation, electrical activity of the heart, electrical activity of other muscles, electrical activity of the brain, pulse rate, blood pressure, blood oxygen saturation level, skin temperature, and core temperature.

The term “electrode”, as used herein, means and includes, without limitation, any electrical conductor used to provide physical communication between the metallic and non-metallic materials and/or compositions that comprise a circuit.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

Although the ECG physiological monitoring systems and associated methods of the invention may be described in connection with monitoring ECG signals representing a defined physiological characteristic, e.g. heart rate, it is understood that the ECG physiological monitoring systems and associated methods of the invention can be employed to monitor ECG signals representing various physiological characteristics.

It is further understood that although the ECG physiological monitoring systems and associated methods of the invention are described herein in connection with a wearable garment, bed and car seat, the invention is not limited to such use/applications. The physiological monitoring systems and associated methods of the invention can also be employed in any other apparatus or system that maintains physical communication with a subject.

It is also understood that although the physiological monitoring systems and associated methods of the invention are described herein in connection with monitoring signals that are representative of physiological characteristics of a human, the invention is not limited to such use. The physiological monitoring systems and associated methods of the invention can also be employed to monitor signals that are representative of physiological characteristics of a non-human, i.e. animal.

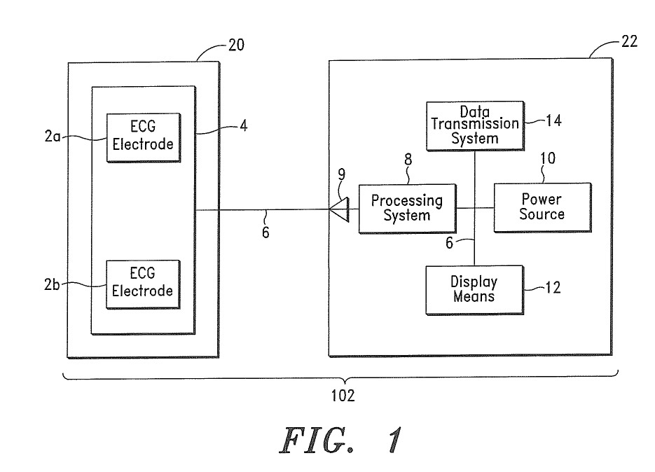

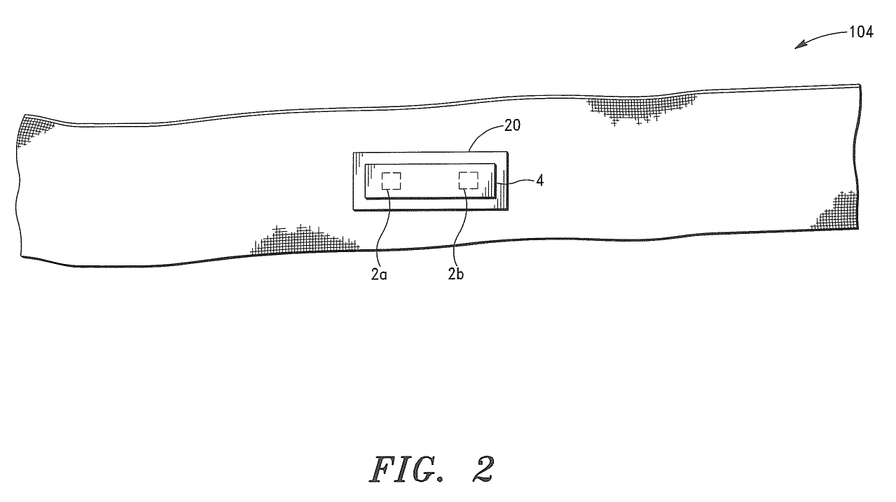

Referring now to FIGS. 1 and 2, in one embodiment of the invention, the ECG physiological monitoring system (denoted “102”) includes an ECG sensor system (or assembly) 20, signal transmission conductors 6 and an electronics module 22. As illustrated in FIGS. 1 and 2, the ECG sensor system 20 further includes first and second ECG electrodes 2a, 2b and reference electrode 4.

In a preferred embodiment, the first and second ECG electrodes 2a, 2b comprise capacitive electrodes that are designed and configured to detect ECG signals without direct physical communication with the skin of a subject.

In a preferred embodiment, the first and second ECG electrodes 2a. 2b, i.e. capacitive electrodes, comprise a conductive material, including, without limitation, a conductive fabric and flexible conductive polymer, e.g., silver coated polyesters, carbon-impregnated polymers.

In some embodiments, the first and second ECG electrodes 2a, 2b preferably comprise an area in the range of 1.0-10 cm².

Preferably, the first and second ECG electrodes 2a, 2b, i.e. capacitive electrodes, are configured to couple to a subject's skin and accurately detect ECG signals at electrode-to-skin distances in the range of 0.1-1.0 mm.

In a preferred embodiment, the first and second ECG electrodes 2a, 2b are configured to electrically couple to a subject's skin and accurately detect ECG signals at electrode-to-skin distances in the range of 0.1-1.0 mm.

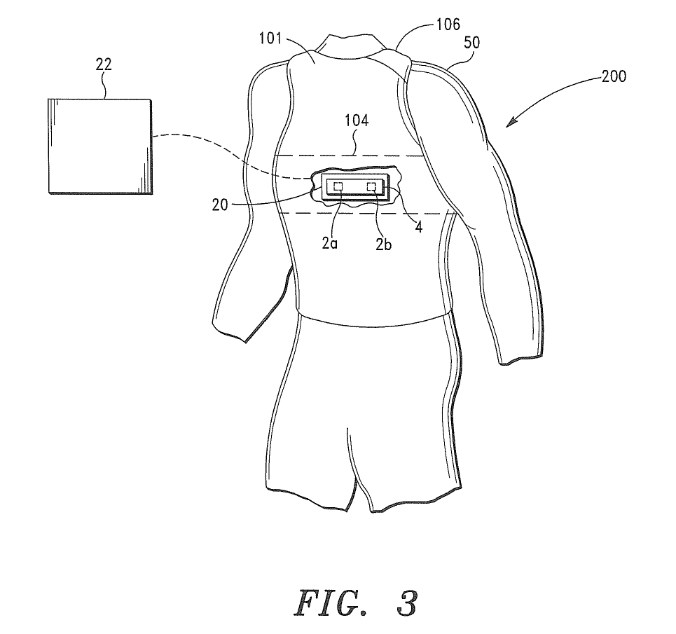

In some embodiments, the reference electrode 4 preferably comprises a conductive material that is incorporated into a garment band 104, which is configured to wrap around a subject or to be incorporated into a wearable physiological monitoring system. (See FIGS. 2 and 3)

According to the invention, the conductive material can comprise, without limitation, silver coated polyesters, carbon-impregnated polymers and conductive rubbers. Preferably, the conductive material comprises a flexible and/or stretchable configuration.

In a preferred embodiment, the conductive material is configured to move or stretch along at least two axes.

According to the invention, the reference electrode 4 can comprise any shape or dimension required to shield the first and second ECG electrodes 2a, 2b.

In some embodiments, the reference electrode 4 preferably comprises an area in the range of two to twenty times (2-20×) the area of the first and second ECG electrodes 2a, 2b.

According to the invention, the reference electrode 4 provides a capacitive reference as a function of the capacitance potential of the first and second ECG electrodes 2a, 2b. In a preferred embodiment, the capacitive reference comprises the average of the capacitance potential of the first and second ECG electrodes 2a, 2b (C_{Avg}).

In some embodiments, the reference electrode 4 is configured to provide an average the capacitance potential (C_{Avg}) of any given quantity of ECG electrodes.

In some embodiments, the reference electrode 4 is also configured to average the capacitance potential noise generated by the anatomy of a subject, e.g., skin, muscle, etc., and the capacitance potential of the first and second ECG electrodes 2a, 2b to provide an average capacitance potential value (C′_{Avg}).

According to the invention, the average capacitance potential values C_{Avg}, C′_{Avg} represent the ECG signals associated with a subject.

In some embodiments, the reference electrode 4 provides an electromagnetic shield for the first and second ECG electrodes 2a, 2b.

According to the invention, the reference electrode 4 reduces external electromagnetic interference, such as interference due to the magnetic fields of power lines, by shielding the first and second ECG electrodes 2a, 2b. By positioning the first and second ECG electrodes 2a, 2b between the reference electrode 4 and the skin of a monitored subject, signal interference due to the displacement of the electrode-to-skin distance is also reduced.

According to the invention, the ECG sensor system 20 can comprise any number of ECG electrodes and reference electrodes. In some embodiments, the ECG sensor system 20 thus comprises a single ECG electrode. In some embodiments, the ECG sensor system 20 comprises a plurality of ECG electrodes.

In some embodiments, the ECG sensor system 20 comprises a single reference electrode. In some embodiments, the ECG sensor system 20 comprises a plurality of reference electrodes.

As illustrated in FIG. 1, in a preferred embodiment of the invention, the ECG sensor system 20 comprises two ECG electrodes and one reference electrode.

Referring back to FIG. 1, in a preferred embodiment, the physiological monitoring system 102 further includes an electronics module 22. The electronics module 22 preferably includes processing system 8, which is programmed and configured to control the ECG sensor system 20 and the function thereof, and the transmission and receipt of signals therefrom.

The processing system 8 is also preferably programmed and configured to receive and process transmissions or signals from the ECG sensor system 20, and determine physiological information associated with a monitored subject (as a function of the signals), including at least one physiological characteristic, e.g. heart rate.

In some embodiments of the invention, the electronics module 22 comprises a power source 10. According to the invention, the power source 10 can comprise any system configured to provide electrical energy to a device, such as a battery.

In some embodiments of the invention, the electronics module 22 further includes a data transmission system 14. Preferably, the data transmission system 14 comprises a transmitter that is programmed and configured to wirelessly transmit processed signals representing physiological characteristics to a remote signal receiving device (not shown), e.g., a base module or a hand-held electronic device, such as a smart phone, tablet, computer, wearable electronic, etc.

In some embodiments of the invention, the physiological monitoring system 102 is further configured to monitor one or more additional physiological characteristics associated with a monitored subject. Thus, in some embodiments, the physiological monitoring system 102 further includes one or more additional physiological sensors, such as a pulse oximeter (SₚO₂) or core body temperature sensor (not shown), which are in communication with the signal transmission conductors 6.

Referring now to FIGS. 2-6, several embodiments of ECG physiological monitoring systems of the invention will be described in detail.

Referring first to FIGS. 2-4, there is shown one embodiment of a wearable ECG physiological monitoring system 200. As illustrated in FIG. 3, the wearable ECG physiological monitoring system 200 includes garment 101, and the ECG sensor system 20 and electronics module 22 shown in FIG. 1.

According to the invention, the wearable ECG physiological monitoring system 200 can comprise any wearable garment, such as the garments disclosed in U.S. application Ser. No. 13/854,230, which is expressly incorporated by reference herein in its entirety.

As also illustrated in FIG. 3, in the noted embodiment, the wearable garment 101 is preferably configured to cover at least the chest region and upper back of a subject 50. According to the invention, the wearable garment 101 can, however, also be configured to cover other regions of the subject 50, including, without limitation, the lower abdominal region.

As illustrated in FIGS. 2 and 3, in some embodiments, the wearable garment 101 includes band 104, which, as discussed below, is preferably incorporated in the wearable garment 101.

According to the invention, the band 104 can comprise various conventional fabrics having fibers of variable loft and thickness. In some embodiments of the invention, the garment 101 comprises a form fitting garment constructed of Lycra® or like material.

In a preferred embodiment, the band 104 is attached to an interior portion of the wearable garment 101. According to the invention, the band 104 can be permanently attached to the garment 101 or removeably secured to the garment 101, e.g. via a zipper or Velcro® system.

In some embodiments of the invention, at least one of the shoulder portions 106 of the garment 101 comprises a two-piece portion, i.e. an over-lapping strap configuration, to facilitate easy placement of the garment 101 on a wearer, e.g., elderly user. In the noted embodiments, the two-piece portion includes a conventional Velcro® system or hooks or snaps to secure the ends of the over-lapping strap after the garment 101 is positioned on the wearer's body.

As indicated above and shown in FIG. 2, the band 104 includes ECG sensor system 20. In a preferred embodiment, the band 104 is positioned on the garment 101 such that the ECG sensor system 20 is disposed proximate the thoracic region of the subject 50 when the garment is worn by the subject 50.

In some embodiments, the band 104 further comprises integral signal transmission conductors 6. In a preferred embodiment, the signal transmission conductors 6 are disposed in a flexible configuration thereon.

In some embodiments, the signal transmission conductors 6 comprise a conventional insulated conductive wire. such as a copper wire insulated by a rubber coating.

In some embodiments of the invention, the signal transmission conductors 6 comprise conductive fabric.

In some embodiments, the signal transmission conductors 6 comprise a thin linear member, e.g. thread or chord, which is wrapped with a conductive wire. Preferably, the linear member comprises a stretchable member, i.e. is at least partially constructed of a stretchable material, and the wire is spirally wrapped around the stretchable member.

In a preferred embodiment, the wearable ECG physiological monitoring system 200 further includes electronics module 22, which is in direct communication with the ECG sensor system 20 via the signal transmission conductors 6.

In some embodiments, the electronics module 22 is incorporated into and/or disposed on the wearable garment 101. In some embodiments, the electronics module 22 is incorporated into band 104.

As indicated above, in one embodiment of the invention, the electronics module 22 preferably includes at least processing system 8 and data transmission system 14.

As also indicated above, the processing system 8 preferably includes programs, instructions and associated algorithms and parameters to control the ECG sensor system 20 and, hence, the first and second ECG electrodes 2a, 2b and the function thereof, and the transmission and receipt of signals therefrom, as well as the data transmission system 14.

The processing system 8 is also preferably programmed and configured to process signals from the ECG sensor system 20, and determine physiological information associated with the monitored subject (as a function of the signals).

In a preferred embodiment, the processing system is further configured to generate the average capacitance potential values C_{Avg}, C′_{Avg} discussed in detail above.

In some embodiments of the invention, the processing system 8 also includes a “rules set” that includes a rule in which an alert signal is transmitted if the signals from the ECG sensor system 20 indicate that an ECG parameter or other physiological parameter that is being monitored is outside a predetermined range.

In some embodiments, the processing system 8 further comprises at least one signal interference and/or noise reducing algorithm, including, without limitation wavelet neural network algorithms, adaptive filtering algorithms, least mean squared algorithms, normalized least mean squared algorithms, sign error least mean squared algorithms, sign data least mean squared algorithms, sign least mean squared algorithms and recursive least mean squared algorithms.

In some embodiments, the electronics module 22 further comprises a signal filtering means, such as a bandpass filter.

Referring back to FIG. 1, in a preferred embodiment, the electronic module 22 further comprises an amplifier system 9 that is designed and configured to amplify ECG signals, i.e. capacitance signals, provided by the ECG sensor system and transmit the amplified ECG signals to the processing system 8.

In some embodiments, the amplifier system 9 comprises an input impedance in the range of 10⁷ Ω to 10⁹ Ω (100 MΩ−10 GΩ).

In some embodiments, the data transmission system 14 includes a transmitter that is programmed and configured to wirelessly transmit processed signals representing physiological characteristics to a remote signal receiving device, e.g., a base module or a hand-held electronic device, such as a smart phone, tablet, computer, electronic wearable, etc.

In some embodiments, the electronics module 22 further comprises display means 12 and is programmed and configured to display received and/or processed signals.

In some embodiments, the wearable ECG physiological monitoring system 200 further includes one or more additional physiological sensors, such as a temperature or SpO₂ sensor. In at least one embodiment, the system includes a temperature sensor.

According to the invention, the wearable ECG physiological monitoring system 200 can further include a portal, such as a website accessible over a network, to display and store the processed ECG signals.

According to the invention, when the wearable ECG physiological monitoring system 200 is positioned on a subject, the system 200 accurately monitors ECG signals and characteristics, i.e. electrical activity associated with the subject, determines at least one physiological characteristic represented by the ECG signals, and, in some embodiments, wirelessly transmits signals representing the physiological characteristic to a remote receiving and/or display device.

As indicated above, in some embodiments, the garment band 104 illustrated in FIG. 2 and discussed in detail above is configured to wrap around a subject. Thus, a further embodiment of a wearable ECG physiological monitoring system of the invention comprises the garment band 104 and an external or remote electronics module 22. The noted system would similarly accurately monitor ECG signals and characteristics, determine at least one physiological characteristic represented by the ECG signals, and transmit signals representing the physiological characteristic to a remote receiving and/or display device.

By virtue of the dry capacitive ECG electrodes, i.e. first and second ECG electrodes 2a, 2b, the reference electrode 4 material and configuration, the defined spacing by and between the first and second ECG electrodes 2a. 2b and reference electrode 4, and positioning of the monitored subject, the wearable ECG physiological monitoring systems described above provide several unexpected superior results and advantages:

The provision of wearable ECG physiological monitoring systems and methods that are suitable for long term, highly accurate monitoring of physiological characteristics. As discussed above, in a preferred embodiment, accurate monitoring of physiological characteristics is achieved by averaging the capacitive potential of ECG electrode(s) over time.
The provision of wearable ECG physiological monitoring systems and methods that substantially reduce or abate external electromagnetic interference, e.g. electromagnetic radiation from any powered device, power lines and interstellar sources. As discussed above, in a preferred embodiment, the external electromagnetic interference reduction/abatement is achieved by utilizing a reference electrode as an electromagnetic shield for the ECG electrodes.
The provision of wearable ECG physiological monitoring systems and methods that substantially reduce or abate internal interference, e.g. interference due to changes in displacement of the electrode-to-skin distance and friction between the electrodes and the medium that the electrodes are attached to.
The provision of wearable ECG physiological monitoring systems and methods that provide a substantially greater (or enhanced) signal-to-noise (SNR) ratio compared to conventional ECG physiological monitoring systems.

As indicated above, according to the invention, the ECG physiological monitoring systems of the invention can also be employed in other apparatuses and/or systems that maintain direct physical contact with a subject.

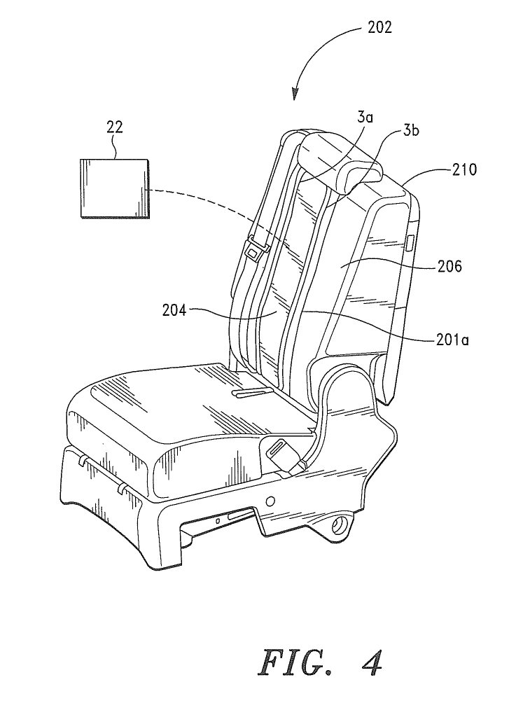

Referring now to FIG. 4, there is shown another embodiment of an ECG physiological monitoring system of the invention. As illustrated in FIG. 4, the system 202 comprises a seat 210 having an integrated ECG sensor system 201a, which is disposed on a seat surface 206, and an external or remote electronics module 22.

According to the invention, the electronics module 22 can also integrated into the vehicle seat 210.

As further illustrated in FIG. 4, the integrated ECG sensor system 201a comprises first and second ECG electrode strips 3a, 3b that are positioned on a reference electrode surface 204.

In a preferred embodiment, the first and second ECG electrode strips 3a, 3b comprise one of the aforementioned ECG electrodes. In some embodiments, the reference electrode surface 204 comprises one of the aforementioned reference electrodes.

According to the invention, the ECG electrode strips can comprise any width and any length, thus, in some embodiments the ECG electrode strips comprise a width in the range of 1.0-5.0 cm and a length in the range of 1.0-100 cm.

According to the invention, the first and second ECG electrode strips 3a, 3b and reference electrode surface 204 are preferably in direct communication with a subject, wherein the reference electrode surface 204 provides a capacitive reference based on the first and second ECG electrode strips 3a, 3b. In a preferred embodiment, the capacitive reference similarly comprises the average of the capacitance potential of the first and second ECG electrodes 3a, 3b (C_{Avg}).

As discussed in detail below, in some embodiments, the reference electrode surface 204 is configured to average the capacitance potential of multiple ECG electrode strips.

In some embodiments, the reference electrode surface 204 is also configured to similarly average the capacitance potential noise generated by the anatomy of a subject, e.g., skin, muscle, etc., and the capacitance potential of the first and second ECG electrode strips 3a. 3b to provide an average capacitance potential value (C′_{Avg}).

As indicated above, the average capacitance potential values C_{Avg}, C′_{Avg} represent ECG signals associated with a subject.

According to the invention, when a subject is seated in seat 210, the ECG physiological monitoring system 202 similarly accurately monitors ECG signals and characteristics associated with the subject, determines at least one physiological characteristic represented by the ECG signals, and transmits signals representing the physiological characteristic to a remote receiving and/or display device.

As will be readily appreciated by one having skill in the art, the ECG physiological monitoring system 202 can be employed with any conventional seating means, e.g. chairs, sofas, airline seats, etc.

Again, by virtue of the use of the dry capacitive ECG electrodes, i.e. first and second ECG electrode strips 3a, 3b, the reference electrode surface 204 material and configuration, the defined spacing by and between the first and second ECG electrode strips 3a, 3b and reference electrode 204, and positioning of the monitored subject, the ECG physiological monitoring system 202 described above similarly provides the superior results and advantages provided by the wearable ECG physiological monitoring system 200.

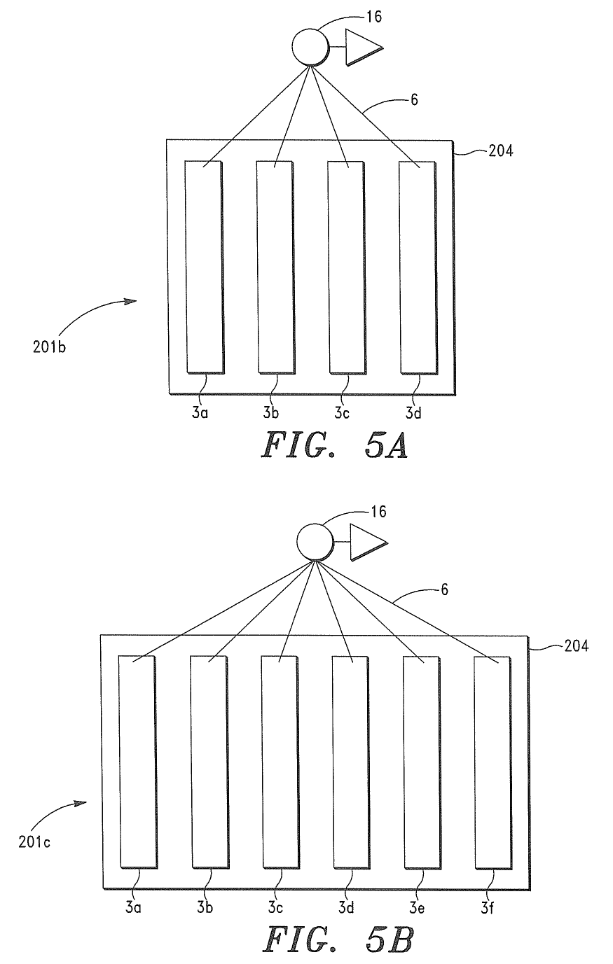

According to the invention, the integrated ECG sensor system 201a described above can comprise more than two ECG electrode strips. Illustrative are the ECG sensor systems 201b and 201c shown in FIGS. 5A and 5B.

Referring now to FIG. 5A, there is shown one embodiment of an integrated ECG sensor system 201b comprising first, second, third and fourth ECG electrode strips 3a, 3b, 3c, 3d and reference electrode surface 204. As illustrated in FIG. 5A, ECG electrode strips 3a, 3b, 3c, 3d are in direct communication with amplifier system 16 via signal transmission conductors 6.

Referring now to FIG. 5B, there is shown another embodiment of an integrated ECG sensor system 201c similarly comprising first, second, third, fourth, fifth and sixth ECG electrode strips 3a, 3b, 3c, 3d, 3e, 3f and reference electrode surface 204. As illustrated in FIG. 5B, ECG electrode strips 3a, 3b, 3c, 3d, 3e, 3f are in direct communication with amplifier system 16 via signal transmission conductors 6.

Referring back to FIG. 4, according to the invention, the first and second ECG electrode strips 3a, 3b and reference electrode surface 204 are preferably in direct communication with a subject, wherein the reference electrode surface 204 provides a capacitive reference based on the first and second ECG electrode strips 3a, 3b. In a preferred embodiment, the capacitive reference similarly comprises the average of the capacitance potential of the first and second ECG electrodes 3a, 3b (C_{Avg}).

In some embodiments, the reference electrode surface 204 is similarly configured to average the capacitance potential noise generated by the anatomy of a subject, e.g., skin, muscle, etc., and the capacitance potential of the first and second ECG electrodes 3a, 3b to provide an average capacitance potential value (C′_{Avg}).

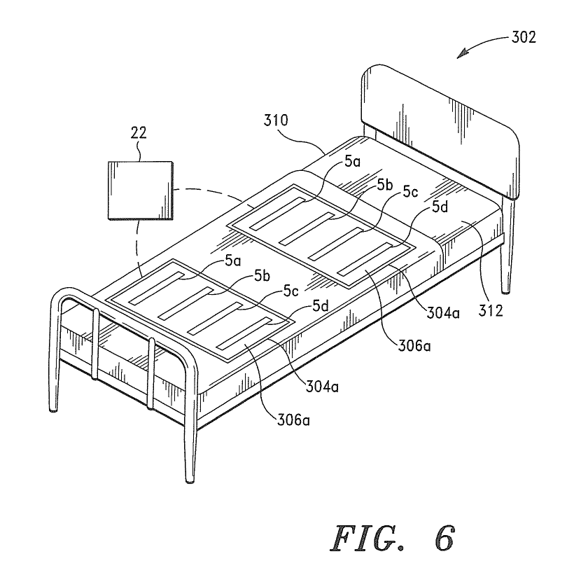

Referring now to FIG. 6, there is shown another embodiment of an ECG physiological monitoring system of the invention. As illustrated in FIG. 6, the physiological monitoring system 302 comprises a bed 310 having two ECG sensor systems 304a and electronics module 22.

According to the invention, the ECG sensor systems can be placed in any position on the bed surface 312. In a preferred embodiment, one ECG sensor system 304a is positioned at one end of the bed 310 and one ECG sensor system 304a is disposed on the other end of bed 310.

According to the invention, the electronics module 22 can also be integrated into the bed 310.

As illustrated in FIG. 6, in the illustrated embodiment, ECG sensor system 304a comprises first, second, third and fourth ECG electrode strips 5a, 5b, 5c, 5d and reference electrode layer 306a.

Although, in the illustrated embodiment, the ECG sensor systems 304a comprise similar systems, according to the invention, dissimilar ECG systems can also be employed, e.g., one ECG sensor system comprising four ECG electrode strips and one ECG sensor system comprising six ECG electrode strips.

Preferably, the ECG electrode strips 5a, 5b, 5c, 5d comprise one of the aforementioned ECG electrodes. In some embodiments, the reference electrode layer 306a comprises one of the aforementioned reference electrodes.

According to the invention, when a subject is in a supine or prone position on the bed 310 such that the ECG sensor systems 304a and, hence, ECG electrode strips 5a, 5b, 5c, 5d and the reference electrode 306a are covered by the subject's body, the ECG physiological monitoring system 302 similarly accurately monitors ECG signals and characteristics associated with the subject, determines at least one physiological characteristic represented by the ECG signals, and transmits signals representing the physiological characteristic to a remote receiving and/or display device.

By virtue of the use of the dry capacitive ECG electrodes, i.e. ECG electrode strips 5a, 5b, 5c, 5d and the reference electrode 306a material and configuration, the defined spacing by and between the ECG electrode strips 5a, 5b, 5c, 5d and reference electrode 306a, and positioning of the monitored subject, the ECG physiological monitoring system 302 described above similarly provides the superior results and advantages provided by the wearable ECG physiological monitoring system 200.

As will readily be appreciated by one having ordinary skill in the art, the present invention provides numerous advantages compared to prior art methods and systems for monitoring and/or detecting physiological characteristics. Among the advantages are the following:

The provision of ECG physiological monitoring systems and methods that accurately monitor ECG signals representing one or more physiological characteristics associated with a user or wearer.
The provision of ECG physiological monitoring systems and methods that accurately measure one or more additional physiological characteristics associated with a user or wearer, e.g. body temperature.
The provision of ECG physiological monitoring systems and methods that do not require the use of conductive gels.
The provision of ECG physiological monitoring systems that are suitable for long term monitoring of physiological characteristics.
The provision of ECG physiological monitoring systems and methods that substantially reduce or abates external electromagnetic interference.
The provision of ECG physiological monitoring systems and methods that substantially reduce or abate internal interference.
The provision of ECG physiological monitoring systems and methods that provide a substantially greater (or enhanced) signal-to-noise (SNR) ratio compared to conventional ECG physiological monitoring systems.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An electrocardiography (ECG) physiological monitoring system, comprising:
an ECG sensor assembly that is configured to be positioned proximate to a subject's skin;
said ECG sensor assembly comprising at least first and second capacitive electrodes configured to electrically couple to said subject's skin and detect ECG signals, said ECG sensor assembly comprising at least a first reference electrode that is configured to average the capacitance potential of said first and second electrodes;
an electronics module comprising a processing system and a data transmission system, said electronics module being in direct communication with said ECG sensor assembly, said processing system including programs, instructions and associated algorithms and parameters to control said ECG sensor assembly, retrieve and process ECG signals transmitted by said ECG sensor assembly, determine physiological information associated with said subject as a function of said ECG signals, said data transmission system including a transmitter that is configured to wirelessly transmit said processed ECG signals; and
signal transmission conductors that are configured to provide a signal communication path between said electronics module and said ECG sensor assembly.

2. The ECG physiological monitoring system of claim 1, wherein said first and second capacitive electrodes are positioned between said subject's skin and said first reference electrode, wherein said first reference electrode is configured to shield said first and second capacitive electrodes from electromagnetic interference.

3. The ECG physiological monitoring system of claim 1, wherein said electronics module comprises an amplifier system that is configured to amplify said ECG signals.

4. The ECG physiological monitoring system of claim 1, wherein said system includes a remote display device that is configured to receive and display said processed ECG signals from said electronics module.

5. The ECG physiological monitoring system of claim 1, wherein said system includes at least one additional physiological sensor that is in communication with said signal transmission conductors.
